SEQUENCE LISTING (40 sequences)

SEQ ID NO: 1 - RNA, 21 nt, synthetic construct
  source 1..21: mol_type = other RNA
cgttgtaggaggaatctgttt

SEQ ID NO: 2 - RNA, 21 nt, synthetic construct
  source 1..21: mol_type = other RNA
acagattcctcctacaacgtt

SEQ ID NO: 3 - RNA, 21 nt, synthetic construct
  source 1..21: mol_type = other RNA
gaggaatctgtgctctacttt

SEQ ID NO: 4 - RNA, 21 nt, synthetic construct
  source 1..21: mol_type = other RNA
agtagagcacagattcctctt

SEQ ID NO: 5 - RNA, 21 nt, synthetic construct
  source 1..21: mol_type = other RNA
ggaatctgtgctctacttatt

SEQ ID NO: 6 - RNA, 21 nt, synthetic construct
  source 1..21: mol_type = other RNA
taagtagagcacagattcctt

SEQ ID NO: 7 - RNA, 21 nt, synthetic construct
  source 1..21: mol_type = other RNA
gctctacttacctctcaattt

SEQ ID NO: 8 - RNA, 21 nt, synthetic construct
  source 1..21: mol_type = other RNA
attgagaggtaagtagagctt

SEQ ID NO: 9 - RNA, 21 nt, synthetic construct
  source 1..21: mol_type = other RNA
gcatacacacgtttttctatt

SEQ ID NO: 10 - RNA, 21 nt, synthetic construct
  source 1..21: mol_type = other RNA
tagaaaaacgtgtgtatgctt

SEQ ID NO: 11 - RNA, 21 nt, synthetic construct
  source 1..21: mol_type = other RNA
cccaatgtaccagtatttatt

SEQ ID NO: 12 - RNA, 21 nt, synthetic construct
  source 1..21: mol_type = other RNA
taaatactggtacattgggtt

SEQ ID NO: 13 - RNA, 21 nt, synthetic construct
  source 1..21: mol_type = other RNA
gcttaaactcacacaacaatt

SEQ ID NO: 14 - RNA, 21 nt, synthetic construct
  source 1..21: mol_type = other RNA
ttgttgtgtgagtttaagctt

SEQ ID NO: 15 - RNA, 21 nt, synthetic construct
  source 1..21: mol_type = other RNA
gaccctaaatccaatagaatt

SEQ ID NO: 16 - RNA, 21 nt, synthetic construct
  source 1..21: mol_type = other RNA
ttctattggatttagggtctt

SEQ ID NO: 17 - RNA, 21 nt, synthetic construct
  source 1..21: mol_type = other RNA
cttgttgaggcatttagcttt

SEQ ID NO: 18 - RNA, 21 nt, synthetic construct
  source 1..21: mol_type = other RNA
agctaaatgcctcaacaagtt

SEQ ID NO: 19 - RNA, 21 nt, synthetic construct
  source 1..21: mol_type = other RNA
ggcatcttctacatgtttttt

SEQ ID NO: 20 - RNA, 21 nt, synthetic construct
  source 1..21: mol_type = other RNA
aaaacatgtagaagatgcctt

SEQ ID NO: 21 - RNA, 21 nt, synthetic construct
  source 1..21: mol_type = other RNA
ctgagaaaagccgatattttt

SEQ ID NO: 22 - RNA, 21 nt, synthetic construct
  source 1..21: mol_type = other RNA
aaatatcggcttttctcagtt

SEQ ID NO: 23 - RNA, 21 nt, synthetic construct
  source 1..21: mol_type = other RNA
gagaagcctttacaggtggtt

SEQ ID NO: 24 - RNA, 21 nt, synthetic construct
  source 1..21: mol_type = other RNA
ccacctgtaaaggcttctctt

SEQ ID NO: 25 - RNA, 21 nt, synthetic construct
  source 1..21: mol_type = other RNA
aggagatgatccggcaacttt

SEQ ID NO: 26 - RNA, 21 nt, synthetic construct
  source 1..21: mol_type = other RNA
agttgccggatcatctccttt

SEQ ID NO: 27 - RNA, 21 nt, synthetic construct
  source 1..21: mol_type = other RNA
cagagcaaggaatgtgacttt

SEQ ID NO: 28 - RNA, 21 nt, synthetic construct
  source 1..21: mol_type = other RNA
agtcacattccttgctctgtt

SEQ ID NO: 29 - RNA, 21 nt, synthetic construct
  source 1..21: mol_type = other RNA
gcaaggaatgtgacttccatt

SEQ ID NO: 30 - RNA, 21 nt, synthetic construct
  source 1..21: mol_type = other RNA
tggaagtcacattccttgctt

SEQ ID NO: 31 - RNA, 21 nt, synthetic construct
  source 1..21: mol_type = other RNA
gaatgtgacttccagaccatt

SEQ ID NO: 32 - RNA, 21 nt, synthetic construct
  source 1..21: mol_type = other RNA
tggtctggaagtcacattctt

SEQ ID NO: 33 - RNA, 25 nt, synthetic construct
  source 1..25: mol_type = other RNA
cccaagggctaccatgccaacttct

SEQ ID NO: 34 - RNA, 25 nt, synthetic construct
  source 1..25: mol_type = other RNA
agaagttggcatggtagcccttggg

SEQ ID NO: 35 - RNA, 21 nt, synthetic construct
  source 1..21: mol_type = other RNA
aactattgcttcagctccatt

SEQ ID NO: 36 - RNA, 21 nt, synthetic construct
  source 1..21: mol_type = other RNA
tggagctgaagcaatagtttt

SEQ ID NO: 37 - RNA, 21 nt, synthetic construct
  source 1..21: mol_type = other RNA
gcagagtacacacagcatatt

SEQ ID NO: 38 - RNA, 21 nt, synthetic construct
  source 1..21: mol_type = other RNA
tatgctgtgtgtactctgctt

SEQ ID NO: 39 - RNA, 22 nt, synthetic construct
  source 1..22: mol_type = other RNA
ggctctagaaaagcctatgctt

SEQ ID NO: 40 - RNA, 22 nt, synthetic construct
  source 1..22: mol_type = other RNA
gcataggcttttctagagcctt

(19) United States
(12) Patent Application Publication
(10) Pub. No.: US 2025/0011788 A1
(43) Pub. Date: Jan. 9, 2025

(54) NUCLEIC ACID INTERFERENCE PHARMACEUTICAL COMPOSITION, AND DRUG FOR TREATING COLORECTAL CANCER, GASTRIC CANCER, AND PROSTATE CANCER

(71) Applicant: SIRNAOMICS BIOPHARMACEUTICALS (SUZHOU) CO., LTD., Suzhou (CN)

(72) Inventors: Deling Wang, Suzhou (CN); Linying Su, Suzhou (CN); Zhiyuan Wang, Suzhou (CN); Jin Zhang, Suzhou (CN); Xilong Liu, Suzhou (CN); Cheng Zhou, Suzhou (CN); Jun Xu, Suzhou (CN); Patrick Y Lu, Suzhou (CN)

(73) Assignee: SIRNAOMICS BIOPHARMACEUTICALS (SUZHOU) CO., LTD., Suzhou (CN)

(21) Appl. No.: 18/706,229

(22) PCT Filed: Mar. 16, 2023
(86) PCT No.: PCT/CN2023/081796

§ 371 Date: Apr. 30, 2024

(30) Foreign Application Priority Data
Mar. 17, 2022 (CN) 202210263654.8

(51) Int. Cl. C12N 15/113; A61K 47/42; A61P 35/00

(52) U.S. Cl. CPC C12N 15/1136; A61K 47/42; A61P 35/00; C12N 15/1138; C12N 2310/14

(57) ABSTRACT

Provided herein are nucleic acid interference pharmaceutical compositions, and drugs for treating colorectal cancer, gastric cancer and prostate cancer. For two targets (TGF-β1 and MyD88) related to tumor microenvironment and tumor metastasis in colorectal cancer, gastric cancer, and prostate cancer, an MyD88/TGF-β1 dual-target nucleic acid interference pharmaceutical composition (composition number STP500) is designed and selected, which includes an active ingredient and a pharmaceutically acceptable carrier. The active ingredient includes a first active ingredient capable of inhibiting and silencing the expression of the MyD88 gene and a second active ingredient capable of inhibiting and silencing the expression of TGF-β1 gene. In vivo and in vitro experiments show that the MyD88/TGF-β1 dual-target nucleic acid interference pharmaceutical composition can effectively inhibit the growth of tumor cells in colorectal cancer, gastric cancer, and prostate cancer, and has application for the treatment of colorectal cancer, gastric cancer, and prostate cancer.

### CROSS-REFERENCE TO RELATED APPLICATIONS

This application is a U.S. National Stage application of International Patent application PCT/CN2023/081796, filed Mar. 16, 2023, which claims priority to Chinese application 202210263654.8, filed Mar. 17, 2022, all of which are hereby incorporated by reference in their entirety.

### TECHNICAL FIELD

The present disclosure relates to the field of biomedical technology, in particular to a nucleic acid interference pharmaceutical composition and a drug for the treatment of colorectal cancer, gastric cancer and prostate cancer.

### BACKGROUND

According to the World Health Organization (WHO) statistics (gco.iarc.fr/), the top five cancers with the highest incidence of new cases globally in 2020 included breast cancer (11.7%), lung cancer (11.4%), colorectal cancer (10%), prostate cancer (7.3%) and gastric cancer (5.6%); and the top five cancers with the highest mortality rates included lung cancer (18%), colorectal cancer (9.4%), liver cancer (8.3%), gastric cancer (7.7%) and breast cancer (6.9%).

Colorectal cancer has a relatively high incidence in China and ranks third in the incidence of various malignancies in the world, for which the major cause of death is the invasion and metastasis of cancer. Due to the lack of early diagnosis and efficient screening methods, most patients with colorectal cancer have already progressed to advanced or locally advanced stage at diagnosis, and have a poor prognosis. Tumor metastasis of colorectal cancer is a complex process with multiple steps, stages and gene mutations. For example, tumor cells detach from the primary site and interact with the surrounding stroma, then migrate into the circulatory system and lymphatic system, adhere to the endothelial cell wall, and gradually extend to blood vessels or a larger area, resulting in vascular proliferation, which eventually leads to the formation of new metastases.

The Gastrointestinal tract has the largest and most complex microecosystem in the human body. Gastrointestinal cancers are among the five cancers with the highest incidence and mortality. It is extremely challenging to maintain the highly complex and dynamic microbial balance in the gastrointestinal tract and eliminate cancer cells during the treatment of such cancers. A recent study reported that patients with gastrointestinal cancer-related cachexia had significant weight loss at least 6 months before diagnosis; and most patients diagnosed with gastrointestinal cancers are complicated with cachexia syndrome, regardless of pre-diagnostic weight change and disease stage. Available treatment options for gastrointestinal cancers are relatively limited. According to the Guidelines for the diagnosis and treatment of Colorectal Cancer 2021 issued by the Chinese Society of Clinical Oncology (CSCO) in 2021, the treatment options for colorectal cancer are still based on conventional surgery combined with adjuvant chemotherapy and monoclonal antibody therapy (such as EGFR-targeted cetuximab, VEGF-targeted bevacizumab), while only chemotherapy (e.g. oxaliplatin) or palliative care is available for patients with postoperative recurrent colorectal cancer. The major treatment strategy for gastric cancer is also comprehensive therapy based on surgical resection. Despite the improvement of surgery and chemotherapy, the prognosis is very poor for patients with gastric cancer. There are still many uncertainties for some immune checkpoint drugs or targeted drugs (e.g. PD-1/PD-L1, HER2, EGFR, CLDN18.2) in the clinical development stage. As no favorable dual-targeted combination therapy is available, new therapies are urgently needed to fill the gap.

Prostate cancer is one of the most common urologic malignancies in men, ranking fourth in global incidence in 2020 after breast cancer, lung cancer and colorectal cancer. According to the incidence and mortality data of malignancies in 2015 released by China's National Cancer Center in 2019, prostate cancer ranked sixth in incidence and tenth in mortality. The etiology involves a variety of factors, such as heredity, age, and excessive intake of exogenous alcohol. So far, there is no established medical intervention or dietary therapy to prevent prostate cancer. Common pathological subtypes of prostate cancer include adenocarcinoma, intraductal carcinoma, ductal adenocarcinoma, urothelial carcinoma, squamous cell carcinoma, basal cell carcinoma, etc., among which prostate adenocarcinoma predominates, and therefore prostate cancer is usually also known as prostate adenocarcinoma. Major treatment options are also mostly limited to radical resection, radical external beam radiotherapy, surgical or pharmacological castration, combination chemotherapy or other combination therapies, and castration combined with novel endocrine drugs, with no effective first-line targeted drugs, immunotherapeutic drugs, or novel nucleic acid drugs.

Small nucleic acid drugs have inherent advantages over small molecule and antibody drugs and are promising in the treatment of diseases that are refractory to conventional small molecule drugs and antibody drugs. There are currently five approved RNA interference-based drugs, including Alnylam's ONPATTRO® (Patisiran), GIVLAARI® (Givosiran), OXLUMO® (Lumasiran), AMVUTTRA® (Vutrisiran), and Novartis' LEQVIO® (Inclisiran). ONPATTRO® (Patisiran) is the world's first siRNA small nucleic acid drug, which was approved in the United States and the European Union in August 2018 for the treatment of stage 1 or stage 2 Polyneuropathy in adult patients with hereditary ATTR (hATTR) amyloidosis. On Feb. 21, 2023, Alnylam announced that the FDA has accepted the NDA application for Patisiran for the treatment of transthyroxin-mediated amyloid cardiomyopathy. GIVLAARI® (Givosiran) was approved in November 2019 for the treatment of adolescent and adult patients aged 12 years and older with acute hepatic porphyria (AHP). OXLUMO® (Lumasiran) was approved by the FDA in November 2020 for the treatment of patients of all ages with primary hyperoxaluria type 1 (PH1). LEQVIO® (Inclisiran) was approved by the European Commission in December 2020 for the treatment of adult patients with hypercholesterolemia or mixed dyslipidemia. In September 2022, the European Commission approved the NDA application for AMVUTTRA® (Vutrisiran), a subcutaneous injection every 3 months to treat the stage 1 and 2 polyneuropathies in adults with hereditary transthyretin (hATTR)-mediated amyloidosis. Although small nucleic acids are highly promising in drug development, the limitations of their inherent properties and technical bottlenecks have led to challenges in their development and application, such as easy degradation, interferon response, off-target effect, poor penetration, lack of appropriate drug delivery system, etc., leading to the absence of RNA interference-based drugs available for cancer. Once these challenges are overcome, it will bring great opportunities and a broad market for nucleic acid drugs.

The development of nucleic acid drugs for the treatment of one or more cancers with high incidence is a current research priority with highly promising potential for clinical applications.

### SUMMARY

The object of the present disclosure is to provide a RNA interference-based pharmaceutical composition capable of inhibiting the growth of tumor cells of at least one of colorectal cancer, gastric cancer and prostate cancer.

Another object of the disclosure is to provide a drug for the treatment of colorectal and/or gastric and/or prostate cancer.

To achieve the above purpose, the technical scheme adopted by the disclosure is:

A RNA interference-based pharmaceutical composition, which comprises an active substance and a pharmaceutically acceptable carrier, wherein the active substance comprises a first active substance capable of inhibiting and silencing MyD88 gene expression and a second active substance capable of inhibiting and silencing TGF-β1 gene expression.

Specifically, the first active substance is one or more of the siRNA molecules, miRNA molecules, or antisense oligonucleotide molecules capable of binding to mRNA encoding the MyD88 protein and inhibiting its expression.

Specifically, the second active substance is one or more of the siRNA molecules, miRNA molecules, or antisense oligonucleotide molecules capable of binding to mRNA encoding TGF-β1 protein and inhibiting its expression.

The present disclosure provides a TGF-β1/MyD88 dual-target RNA interference-based pharmaceutical composition that includes an active substance and a pharmaceutically acceptable carrier, wherein the active substance includes a first active substance capable of inhibiting and silencing MyD88 gene expression and a second active substance capable of inhibiting and silencing TGF-β1 gene expression.

Further, the active substance may be of an siRNA molecule targeting the MyD88 gene and an siRNA molecule targeting the TGF-β1 gene.

The siRNA molecule targeting the MyD88 gene may be an oligonucleotide with a chain length of 17-28 base pairs. For example, siRNA molecules are 17, 18, 19, 20, 21, 22, 23, 24, 25, 26, 27, or 28 base pairs long.

The siRNA molecule targeting the TGF-β1 gene may be an oligonucleotide with a chain length of 17-28 base pairs. For example, siRNA molecules are 17, 18, 19, 20, 21, 22, 23, 24, 25, 26, 27, or 28 base pairs long.

According to some embodiments, the first active substance is a siRNA molecule targeting the MyD88 gene, comprising one or more of the following oligonucleotides:
Sense Strand: CGUUGUAGGAGGAAUCUGUdTdT (SEQ ID NO: 1), Antisense Strand: ACAGAUUCCUCCUACAACGdTdT (SEQ ID NO: 2);
Sense Strand: GAGGAAUCUGUGCUCUACUdTdT (SEQ ID NO: 3), Antisense Strand: AGUAGAGCACAGAUUCCUCdTdT (SEQ ID NO: 4);
Sense Strand: GGAAUCUGUGCUCUACUUAdTdT (SEQ ID NO: 5), Antisense Strand: UAAGUAGAGCACAGAUUCCdTdT (SEQ ID NO: 6);
Sense Strand: GCUCUACUUACCUCUCAAUdTdT (SEQ ID NO: 7), Antisense Strand: AUUGAGAGGUAAGUAGAGCdTdT (SEQ ID NO: 8);
Sense Strand: GCAUACACACGUUUUUCUAdTdT (SEQ ID NO: 9), Antisense Strand: UAGAAAAACGUGUGUAUGCdTdT (SEQ ID NO: 10);
Sense Strand: CCCAAUGUACCAGUAUUUAdTdT (SEQ ID NO: 11), Antisense Strand: UAAAUACUGGUACAUUGGGdTdT (SEQ ID NO: 12);
Sense Strand: GCUUAAACUCACACAACAAdTdT (SEQ ID NO: 13), Antisense Strand: UUGUUGUGUGAGUUUAAGCdTdT (SEQ ID NO: 14);
Sense Strand: GACCCUAAAUCCAAUAGAAdTdT (SEQ ID NO: 15), Antisense Strand: UUCUAUUGGAUUUAGGGUCdTdT (SEQ ID NO: 16);
Sense Strand: CUUGUUGAGGCAUUUAGCUdTdT (SEQ ID NO: 17), Antisense Strand: AGCUAAAUGCCUCAACAAGdTdT (SEQ ID NO: 18);
Sense Strand: GGCAUCUUCUACAUGUUUUdTdT (SEQ ID NO: 19), Antisense Strand: AAAACAUGUAGAAGAUGCCdTdT (SEQ ID NO: 20);
Sense Strand: CUGAGAAAAGCCGAUAUUUdTdT (SEQ ID NO: 21), Antisense Strand: AAAUAUCGGCUUUUCUCAGdTdT (SEQ ID NO: 22);
Sense Strand: GAGAAGCCUUUACAGGUGGdTdT (SEQ ID NO: 23), Antisense Strand: CCACCUGUAAAGGCUUCUCdTdT (SEQ ID NO: 24);
Sense Strand: AGGAGAUGAUCCGGCAACUdTdT (SEQ ID NO: 25), Antisense Strand: AGUUGCCGGAUCAUCUCCUdTdT (SEQ ID NO: 26);
Sense Strand: CAGAGCAAGGAAUGUGACUdTdT (SEQ ID NO: 27), Antisense Strand: AGUCACAUUCCUUGCUCUGdTdT (SEQ ID NO: 28);
Sense Strand: GCAAGGAAUGUGACUUCCAdTdT (SEQ ID NO: 29), Antisense Strand: UGGAAGUCACAUUCCUUGCdTdT (SEQ ID NO: 30);
Sense Strand: GAAUGUGACUUCCAGACCAdTdT (SEQ ID NO: 31), Antisense Strand: UGGUCUGGAAGUCACAUUCdTdT (SEQ ID NO: 32);
And/or,
The second active substance is a siRNA molecule targeting the TGF-β1 gene, comprising one or more of the following oligonucleotides:
Sense Strand: CCCAAGGGCUACCAUGCCAACUUCU (SEQ ID NO: 33), Antisense Strand: AGAAGUUGGCAUGGUAGCCCUUGGG (SEQ ID NO: 34);
Sense Strand: AACUAUUGCUUCAGCUCCAdTdT (SEQ ID NO: 35), Antisense Strand: UGGAGCUGAAGCAAUAGUUdTdT (SEQ ID NO: 36);
Sense Strand: GCAGAGUACACACAGCAUAdTdT (SEQ ID NO: 37), Antisense Strand: UAUGCUGUGUGUACUCUGCdTdT (SEQ ID NO: 38).

The carrier may be one or more of a polycation binder, a cationic liposome, a cationic micelle, a cationic polypeptide, a cationic polyacetal, a grafted hydrophilic polymer, a polysaccharide molecule, a polyvesicle, an antibody, a polypeptide molecule, or an aptamer.

According to some embodiments, the carrier is a pharmaceutically acceptable histidine-lysine polymer.

Further, the carrier can be an H3K4b histidine-lysine polymer.

According to some embodiments, the carrier is HKP or HKP (+H).

The disclosure also provides the application of the RNA interference-based pharmaceutical composition in the preparation of drugs for the prevention and treatment of colorectal and/or gastric and/or prostate cancer.

The disclosure also provides a drug for the treatment of colorectal and/or gastric and/or prostate cancer, comprising RNA interference-based pharmaceutical composition.

The feed mass ratio of the first active substance and the second active substance can be of 1:0.8 to 1.2, such as 1:0.8, 1:0.9, 1:1, 1:11, 1:1.2.

According to some embodiments, the feed mass ratio of the siRNA molecule targeting the MyD88 gene and the siRNA molecule targeting the TGF-β1 gene is 1:0.8 to 1.2.

The N/P of the carrier and the active substance can be of 2/1 to 6/1, for example, 2/1, 3/1, 4/1, 5/1, 6/1.

Colorectal cancers include adenocarcinoma, adenosquamous carcinoma, and undifferentiated carcinoma.

Gastric cancers include adenocarcinoma, sig-ring cell carcinoma, adeno-squamous cell carcinoma, medullary carcinoma, carcinoid carcinoma, and undifferentiated cell carcinoma.

Prostate cancers include adenocarcinoma, intraductal carcinoma, ductal adenocarcinoma, urothelial carcinoma, squamous cell carcinoma, and basal cell carcinoma.

The drug may be in the form of a nanoparticle.

Further, the size of the nanoparticle can be 50-200 nm, such as 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm.

According to the one embodiment, the drug is named STP500, where the siRNA molecule targeting the MyD88 gene is:
sense strand: GAAUGUGACUUCCAGACCAdTdT (SEQ ID NO: 31), antisense strand: UGGUCUGGAAGUCACAUUCdTdT (SEQ ID NO: 32);
the siRNA molecules targeting the TGF-β1 gene are: sense strand: CCCAAGGGCUACCAUGCCAACUUCU (SEQ ID NO: 33), antisense strand: AGAAGUUGGCAUGGUAGCCCUUGGG (SEQ ID NO: 34); and the carrier is HKP (+H).

The drug can be administered by subcutaneous injection and/or intravenous injection.

The drug can be administered to mammals.

Compared with the conventional drugs prior art, the present disclosure has the following advantages due to the use of the technical scheme:

Targeting on 2 targets (TGF-β1 and MyD88) related to tumor microenvironment of colorectal cancer, gastric cancer or prostate cancer and tumor metastasis, the present disclosure designs and screens a dual target MyD88/TGF-β1 combined RNA interference-based pharmaceutical composition, which shows in vivo experiments that it can effectively inhibit the growth of colorectal cancer, gastric cancer or prostate cancer cells. It has great application prospects in the treatment of colorectal cancer, gastric cancer or prostate cancer.

### BRIEF DESCRIPTION OF THE DRAWINGS

FIG. 1 a schematic view of the MYD88-toll-like receptor signaling pathway.

FIG. 2 shows the expression of MyD88 gene in the cell lines.

FIG. 3 shows the knockdown effect of MyD88 siRNA on the mRNA expression level of the target gene MyD88.

FIG. 4A shows EC50 data (siRNA concentration to produce half of the effective knockdown effect of candidate MyD88 siRNA) in homo cell lines.

FIG. 4B shows EC50 data (siRNA concentration to produce half of the effective knockdown effect of candidate MyD88 siRNA) in murine cell lines.

FIG. 5A depicts knockdown effect of the candidate siRNA on a target gene protein in MDA-MB-231 cells.

FIG. 5B depicts knockdown effect of the candidate siRNA on the target gene protein in RKO cells.

FIG. 5C depicts knockdown effect of the candidate siRNA on the target gene protein in RM-1 cells).

FIG. 6 is a graph showing in vitro cell scratch tests of the candidate MyD88 siRNA.

FIG. 7 shows the effect of the candidate siRNA on cytokine IL-6 secretion at the cellular level.

FIG. 8 shows photographs of formulations of the candidate MyD88 siRNA and its combination with TGF-β1 siRNA.

FIG. 9A depicts tumor growth curves of groups for a subcutaneous tumor graft model of MC38 cells.

FIG. 9B depicts weight change curves of mice of groups for the subcutaneous tumor graft model of MC38 cells.

FIG. 9C depicts tumor photos of groups for the subcutaneous tumor graft model of MC38 cells.

FIG. 9D depicts tumor weight statistics of groups for the subcutaneous tumor graft model of MC38 cells).

FIG. 10A depicts tumor growth curves of groups for a subcutaneous tumor xenograft model of MKN45 cells.

FIG. 10B depicts weight change curves of mice of groups for the subcutaneous tumor xenograft model of MKN45 cells.

FIG. 10C depicts tumor photos of groups for the subcutaneous tumor xenograft model of MKN45 cells.

FIG. 10D depicts tumor weight statistics of groups for the subcutaneous tumor xenograft model of MKN45 cells).

FIG. 11A depicts tumor growth curves of groups for a subcutaneous tumor graft model of RM-1 cells.

FIG. 11B depicts weight change curves of mice of groups for the subcutaneous tumor graft model of RM-1 cells.

FIG. 11C depicts tumor photos of groups for the subcutaneous tumor graft model of RM-1 cells.

FIG. 11D depicts tumor weight statistics of groups for the subcutaneous tumor graft model of RM-1 cells).

FIG. 12A depicts tumor growth curves of groups for a subcutaneous tumor graft model of MC38 cells.

FIG. 12B depicts survival rate curves of mice of groups for the subcutaneous tumor graft model of MC38 cells.

FIG. 12C depicts transcriptome data of the subcutaneous tumor graft model of MC38 cells: up-regulated/down-regulated genes in the treatment group (STP500) versus the control group (NC) (volcano map).

FIG. 12D: transcriptome data of the subcutaneous tumor graft model of MC38 cells: up-regulated/down-regulated genes in the treatment group (STP500) versus the control group (NC) (heat map).

### DETAILED DESCRIPTION

The present disclosure is further described below in connection with examples below. However, the present disclosure invention is not limited to the following examples. The implementation conditions adopted in the examples can be further adjusted according to the different requirements of the specific use, and unspecified implementation conditions are the usual conditions in the industry. The technical features involved in the embodiments of the present disclosure can be combined as long as they do not conflict with each other.

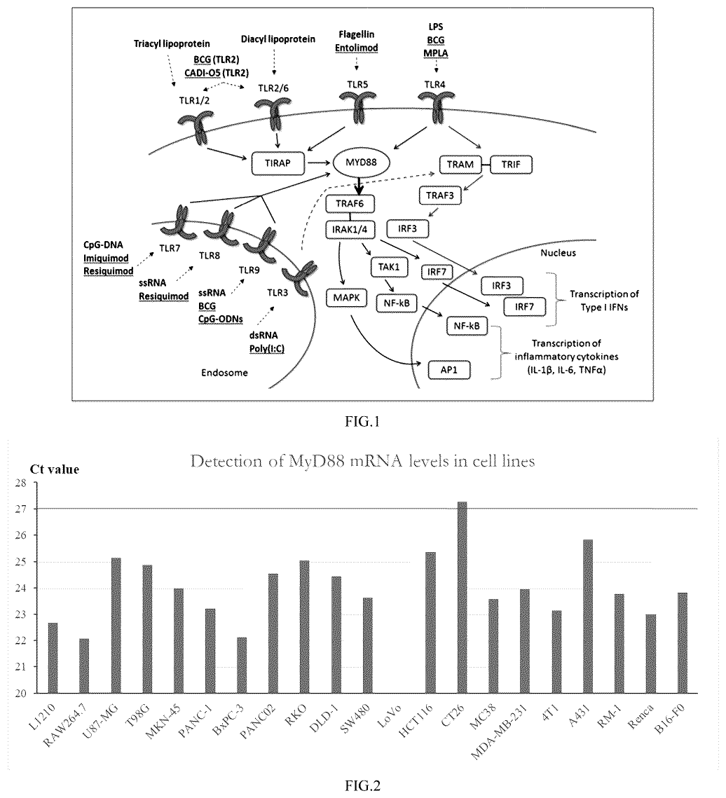

Myeloid Differentiation Factor 88 (MyD88) is a key adaptor molecule in Toll-like receptor (TLR) signaling pathways, which functions in all TLR signaling pathways except TLR3. MyD88 plays an important role in transmission of upstream signals, occurrence and development of diseases as well as mediation of innate immune responses. The TLR family is the most intensively studied class of Pattern recognition receptors (PRRs). It transmits signals into cells by specifically recognizing Pathogen-associated molecular patterns (PAMPs) and thereby regulates downstream signaling cascades. MyD88 is located in the short arm of chromosome 3, and a number of studies have found that the most frequently occurring point mutation is the replacement of leucine (Leu) by proline (Pro) at site 265, which is known as the MYD88 L265P mutation, which leads to the abnormal activation of downstream signaling pathways such as NF-κB. In addition to the NF-κB pathway, MyD88 also regulates immune cells and cytokines (such as IL-6, TNF-α or IL-10) and chemokines by regulating a variety of downstream key molecules through the TLR family, which in turn affects the tumor microenvironment such as Mitogen-active protein kinase (MAPK), Janus kinase and transcription factor 3 (Jak-Stat3) (FIG. 1, image source: doi.org/10.1007/s11523-018-0589-7).

A recent study by Zhang J. et al. in mouse models reported that MyD88 in myoblasts promoted the secretion of Osteopontin (OPN) and promoted M2 polarization of macrophages, leading to activation of STAT3/PPARγ signaling pathway and development of colorectal cancer (CRC). Another study indicated that a MyD88 inhibitor TJ-M2010-5 could be directly used to damage Myeloid-derived suppressor cells (MDSCs) to prevent the development of colitis-related colorectal cancer. This study also suggested that the MyD88 signaling pathway was involved in the regulation of MDSC immunosuppressive function. At the same time, many scholars believe that MyD88 plays a dual role in the occurrence and development of colorectal cancer: a. by enhancing cancer inflammation and intestinal flora imbalance, it induces tumor invasion and self-renewal of tumor cells, and then produces tumor promotion; and b, maintain host-microbiome homeostasis to induce tumor cell cycle arrest and immune responses against cancer cells, resulting in antitumor effects. TLR/IL-1R signaling has also been shown to play a key role in intestinal homeostasis, intestinal inflammation, and colitis-related tumorigenesis by maintaining microbial tolerance of the colon epithelium. MyD88 protein plays a bridging role between TLR/IL-1R inflammation and Ras signaling pathway. Activation of MyD88/TLR/IL-1R signaling pathway leads to activation of Ras/ERK and promotes tumor cell proliferation. Based on these knowledges, the inventors conducted a series of preliminary experimental validations and identified MyD88 as a potential target for the treatment of colorectal cancer.

Transforming Growth Factor-β (TGF-β) signaling pathway plays a key role in the growth, development and differentiation of cells and tissues through a series of signal transmission processes, and also plays important regulatory roles in cell proliferation, intercellular substance generation, differentiation and apoptosis, embryo development, organ formation, immune function, inflammatory response and wound repair. At the moment, A total of 33 TGF-β family proteins are known to exist in humans, including 3 TGF-βs (TGF-β1/2/3), 10 bone morphogenetic proteins (BMPs), and 11 growth and differentiation factors (GDFs), and activin, nodal, inhibin, etc. In cancer cells, the TGF-β signaling pathway is involved in regulating a variety of cellular functions, including cell cycle progression, apoptosis, adhesion, and cell differentiation. In the advanced stage of tumor development, TGF-β promotes cell proliferation, induces angiogenesis, and suppresses immune responses in the tumor microenvironment. As an immunosuppressive cytokine, TGF-β inhibits the development, proliferation, and activation of immune cells, including T cells (CD4⁺ effector T cells and CD8⁺ cytotoxic T cells), NK cells, and macrophages. In addition, TGF-β induces the proliferation of Regulatory T cells (Tregs), which in turn inhibits the activation of effector T cells, NK cells, and macrophages. It can be seen that TGF-β facilitates tumor development by creating an immune-tolerant microenvironment through the suppression of both the innate and adaptive immune systems and promotes tumor metastasis by enhancing the ability of cancer cells to migrate and invade neighboring tissues.

In summary, TGF-β1 signaling pathway has complicated relations with tumorigenesis and metastasis, and the MyD88 protein is a key molecule connecting the upstream TLR family and other inflammatory signaling pathways and cancer RAS pathways. At present, there is no research on the combination of these two pathways in the treatment of gastrointestinal cancer and prostate cancer. The development of the dual-target drug that can effectively target TGF-β1 and MyD88 at the same time will have great application prospects in the treatment of colorectal cancer, gastric cancer and prostate cancer.

Therefore, after a large number of studies and experimental validations, it is found that compared with the MyD88 target used alone or MyD88 target combined with other targets, the combination of TGF-β1 target and MyD88 target has a positive effect of inhibiting the growth of colorectal cancer cells, gastric cancer cells and prostate cancer cells. Furthermore, the inventors proposed a dual-target nucleic acid interference-based pharmaceutical composition that can simultaneously target tumor microenvironment target (MyD88) and tumor transformation and metastasis related target (TGF-β1). A large number of in vivo experiments demonstrate that the dual-target nucleic acid interference-based pharmaceutical composition has excellent performance and higher safety in the treatment of rectal cancer, gastric cancer and prostate cancer, and has great application prospects.

The nucleic acid interference-based pharmaceutical composition provided by the present disclosure includes active ingredients and a pharmaceutically acceptable carrier. The active ingredients include a first active ingredient capable of inhibiting and silencing MyD88 gene expression and a second active ingredient capable of inhibiting and silencing TGF-β1 gene expression.

The first active ingredient is one or more of siRNA molecules, miRNA molecules, or antisense oligonucleotide molecules capable of binding to mRNA encoding a MyD88 protein and inhibiting its expression.

The second active ingredient is one or more of siRNA molecules, miRNA molecules, or antisense oligonucleotide molecules capable of binding to mRNA encoding a TGF-β1 protein and inhibiting its expression.

RNA interference (RNAi) is a universal and effective gene silencing process in the organism kingdom, which widely exists in eukaryotic cells such as plants and animals. RNAi refers to the phenomenon of introducing double strand RNA (dsRNA) into cells to degrade mRNA, resulting in specific gene silencing for genes with sequence homology. Exogenous genes such as transposons, artificial transgenes, viral genes, etc. are randomly integrated into the genome of a host cell. These genes are transcribed by the host cell, often producing some dsRNA, which is usually longer than 30 base pairs. The host cell responds to these dsRNA very quickly, and the endogenous or exogenous dsRNA is cleaved by specific dicers in the body into small double-stranded segments of 21-23 base pairs. These small double-stranded segments are known as small interfering RNAs (siRNAs). The double strands of the siRNAs can be linked with an RNA-induced silencing complex (RISC). After binding with the RISC, targeted cleavage of specific mRNA is realized to interrupt the translation process of the specific mRNA, and inhibit and silence the expression of the target gene.

Unlike siRNA, mature micro RNA (miRNA) is single-stranded RNA, which is processed in vivo by pri-miRNA (primary miRNA) into pre-miRNA (precursor miRNA) in the nucleus, and then transported by an exportin-5 protein from the nucleus to the cytoplasm, where the pre-miRNA is processed by RNase III enzyme Dicer to become mature miRNA. It degrades the target gene mRNA in vivo to regulate its transcription level but not to affect the mRNA stability, and its specificity of binding to mRNA is lower than that of siRNA.

Antisense oligonucleotide (ASO) is a single-stranded DNA molecule that binds to the target gene mRNA according to the base complementary principle, then blocks protein translation through steric hindrance, or degrades the mRNA through cleavage by RNase H, or changes pre-mRNA splicing by interfering with cis-splicing elements, and finally blocks the expression of the target gene.

According to examples, the active ingredients include a siRNA molecule targeting a MyD88 gene and a siRNA molecule targeting a TGF-β1 gene.

The siRNA sequence targeting the MyD88 gene may theoretically bind to and degrade MyD88 mRNA in the target cell through the RNAi mechanism, thereby blocking its protein translation level, and thus inhibiting the protein expression of MyD88.

The siRNA sequence targeting the TGF-β1 gene may theoretically bind to and degrade TGF-β1 mRNA in the target cell through the RNAi mechanism, thereby blocking its protein translation level, and thus inhibiting the protein expression of TGF-β1.

The present disclosure designs a series of siRNA sequences for the target genes TGF-β1 and MyD88 using a specific algorithm and programming with various built-in parameters and conditions. The siRNA molecule targeting the MyD88 gene and the siRNA molecule targeting the TGF-β1 gene are oligonucleotide sequences with a length of 19-25 base pairs, respectively.

The length of the siRNA molecule targeting the MyD88 gene and the length of the siRNA molecule targeting the TGF-β1 gene can be of 21-25 base pairs, respectively.

The main limitation of the nucleic acid interference-based drug entering clinical application is the need for an effective transport carrier (known as introduction/delivery system). The delivery system must be able to effectively protect and transport its cargoes, and must also be able to pass through the cytoplasmic membrane and finally into the cytoplasm after reaching the outside of the target cell, so that the active ingredients of the nucleic acid interference-based drug may play their roles. At present, A variety of delivery systems have been used to target or enhance the delivery of small nucleic acid drugs, such as a polycationic binder, a cationic liposome, a cationic micelle, a cationic polypeptide, a cationic polyacetal, a grafted hydrophilic polymer, a polysaccharide molecule (which forms a polysaccharide-RNA monoconjugate with an RNA molecule), a polyvesicle, an antibody, a polypeptide molecule (which self-assembles to form a polypeptide nanoparticle carrier introduction system), or an aptamer and so on.

According to examples, the present disclosure uses a polypeptide nanoparticle carrier introduction system (a polypeptide introduction system rich in histidine-lysine) of which the independent intellectual property rights are owned by the applicant. For details of this introduction system, reference could be made to Composition and methods of controllable co-coupling polypeptide nanoparticle delivery system for nucleic acid therapeutics (Patent No.: CN 112703196 A).

The delivery carrier can be HKP or HKP(+H).

By means of Peptide Nanoparticle (PNP) technology, the siRNA molecule targeting the MyD88 gene and the siRNA molecule targeting the TGF-β1 gene are encapsulated into the HKP or HKP+H (histidine-lysine polymer) carrier to prepare a nanoparticle formulation.

The feed mass ratio of the siRNA molecule targeting the MyD88 gene and the siRNA molecule targeting the TGF-β1 gene is 1:0.8-1.2, and the N/P of the carrier to the active ingredient is 2/1 to 4/1.

According to the present disclosure, the drug combination encapsulated by the PNP technology and having siRNA co-targeting MyD88 (MD8) and TGF-β1(TF1) introduced therein can significantly inhibit the growth of colorectal cancer, gastric cancer and prostate cancer, and shows potential application prospects in the treatment of colorectal cancer, gastric cancer and prostate cancer.

The technical solution and technical effect of the present disclosure are elaborated by examples as follows.

### Example 1

siRNA Sequences Designed for Two Targets (MyD88 and TGF-β1)

A series of siRNA sequences, including oligonucleotide sequences of 25 base pairs and 21 base pairs in length, were designed for the target genes TGF-β1 and MyD88 using a specific algorithm and programming with various built-in parameters and conditions. The siRNA sequences for the two targets TGF-β1 and MyD88 are listed in Table 1. The characteristics of these sequences include, but are not limited to, targeting gene coding sequences, reasonable thermodynamic stability, and low expected toxic and side effects.

The siRNA sequence targeting the MyD88 gene may theoretically bind to and degrade MyD88 mRNA in the target cell through the RNAi mechanism, thus down-regulating the protein expression of MyD88.

The siRNA sequence targeting the TGF-β1 gene may theoretically bind to and degrade TGF-β1 mRNA in the target cells through RNAi mechanism, thus down-regulating the protein expression of TGF-β1.

TABLE 1
Sequences for candidate MyD88 siRNA and
its combination with TGF-β1 siRNA
and sequences for other control group
siRNA in related experiments

| | siRNA | SS(5′-3′) | AS(5′-3′) |
|---|---|---|---|
| Gene | No.# | SEQ ID NO. | SEQ ID NO. |
| MyD | MD8-21- | CGUUGUAGGAGGAAUCUGU | ACAGAUUCCUCCUACAACGd |
| 88 | h1# | dTdT 1 | TdT 2 |
| | MD8-21- | GAGGAAUCUGUGCUCUACUd | AGUAGAGCACAGAUUCCUCd |
| | h2# | TdT 3 | TdT 4 |
| | MD8-21- | GGAAUCUGUGCUCUACUUAd | UAAGUAGAGCACAGAUUCCd |
| | h3# | TdT 5 | TdT 6 |
| | MD8-21- | GCUCUACUUACCUCUCAAUd | AUUGAGAGGUAAGUAGAGCd |
| | h4# | TdT 7 | TdT 8 |
| | MD8-21- | GCAUACACACGUUUUUCUAd | UAGAAAAACGUGUGUAUGCd |
| | h5# | TdT 9 | TdT 10 |
| | MD8-21- | CCCAAUGUACCAGUAUUUAd | UAAAUACUGGUACAUUGGGd |
| | h6# | TdT 11 | TdT 12 |
| | MD8-21- | GCUUAAACUCACACAACAAd | UUGUUGUGUGAGUUUAAGCd |
| | h7# | TdT 13 | TdT 14 |
| | MD8-21- | GACCCUAAAUCCAAUAGAAd | UUCUAUUGGAUUUAGGGUCd |
| | h8# | TdT 15 | TdT 16 |
| | MD8-21- | CUUGUUGAGGCAUUUAGCU | AGCUAAAUGCCUCAACAAGd |
| | h9# | dTdT 17 | TdT 18 |
| | MD8-21- | GGCAUCUUCUACAUGUUUUd | AAAACAUGUAGAAGAUGCCd |
| | h10# | TdT 19 | TdT 20 |
| | MD8-21- | CUGAGAAAAGCCGAUAUUU | AAAUAUCGGCUUUUCUCAGd |
| | h11# | dTdT 21 | TdT 22 |
| | MD8-21- | GAGAAGCCUUUACAGGUGG | CCACCUGUAAAGGCUUCUCd |
| | hm1# | dTdT 23 | TdT 24 |
| | MD8-21- | AGGAGAUGAUCCGGCAACUd | AGUUGCCGGAUCAUCUCCUd |
| | hm2# | TdT 25 | TdT 26 |
| | MD8-21- | CAGAGCAAGGAAUGUGACU | AGUCACAUUCCUUGCUCUGd |
| | hm3# | dTdT 27 | TdT 28 |
| | MD8-21- | GCAAGGAAUGUGACUUCCAd | UGGAAGUCACAUUCCUUGCd |
| | hm4# | TdT 29 | TdT 30 |
| | MD8-21- | GAAUGUGACUUCCAGACCAd | UGGUCUGGAAGUCACAUUCd |
| | hm5# | TdT 31 | TdT 32 |
| TGF- | TF1-25- | CCCAAGGGCUACCAUGCCAA | AGAAGUUGGCAUGGUAGCC |
| β1 | hm5# | CUUCU 33 | CUUGGG 34 |
| | TF1-21- | AACUAUUGCUUCAGCUCCAd | UGGAGCUGAAGCAAUAGUU |
| | hm3# | TdT 35 | dTdT 36 |
| | TF1-21- | GCAGAGUACACACAGCAUAd | UAUGCUGUGUGUACUCUGCd |
| | h1# | TdT 37 | TdT 38 |
| NC | NC | GGCUCUAGAAAAGCCUAUG | GCAUAGGCUUUUCUAGAGCC |
| | siRNA-1# | CdTdT 39 | dTdT 40 |

### Example 2

### In Vitro Screening of MyD88 Gene Expression in Cell Lines

The expression levels of the MyD88 gene in a variety of human-derived and mouse-derived cell lines were identified by a RT-PCR method. The expression results of the MyD88 gene in cell lines are shown in FIG. 2. The selected cell lines include human-derived ones (Homo): U87-MG, T98G, MKN-45, PANC-1, BxPC3, RKO, DLD-1, SW480, LoVo, HCT116, MDA-MB-231 and A431; and mouse-derived ones (Mus): L1210, Raw264.7, PANC02, CT26, MC38, 4T1, RM-1, Renca and B16-F0.

According to the data in FIG. 2, the MyD88 gene expression levels in all the cell lines except the CT26 cell meet the screening criteria (Ct value is 20-27).

### Example 3

In Vitro Screening of siRNA Sequences Targeting MyD88 Gene (Cellular Level)

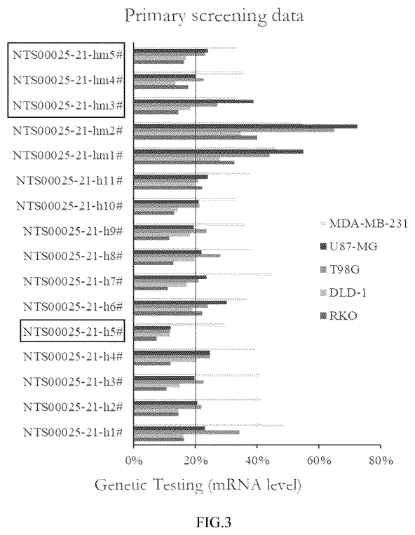

Some cell lines screened out in Example 2 were used for primary siRNA screening, including a breast cancer cell line (MDA-MB-231), a neuroglioma cell line (U87-MG), a human brain glioma cell line (T98G), a human colorectal cancer epithelial cell line (DLD-1) and a human colon cancer cell line (RKO). Specifically, the MyD88 siRNA sequence in Table 1 was transfected into the above five cells by lipo2000, and the transfection concentration was 100 nM (calculated by siRNA). After transfection for 20-24 h, the total RNA of the cells was extracted, and the mRNA expression level of the target gene MyD88 was detected by RT-PCR technology. The results show (FIG. 3) that the target gene silencing effect of all human-derived MyD88 siRNA sequences is higher than 50% (KD>50%), and the sequence with the best effect is MD-21-h5# (NTS00025-21-h5# in FIG. 3). Among the human-mouse homologous sequences, the target gene silencing effect of three siRNA sequences in the five cell lines is higher than 60% (KD>60%), and the target gene silencing effect in colorectal cells is as high as about 85%. The three specifical sequences are MD8-21-hm3# (NTS00025-21-hm3# in FIG. 3), MD8-21-hm4# (NTS00025-21-hm4# in FIG. 3) and MD8-21-hm5# (NTS00025-21-hm5# in FIG. 3).

Then, for these candidate siRNA sequences, multiple concentration gradients were set in human-derived cell lines and mouse-derived cell lines respectively for cell transfection, and their EC50 values (half-maximal effective concentration) were obtained. The experiment results are shown in FIGS. 4A and 4B. The results show that in the human-derived cell lines, except for one sequence whose EC50>10 nM in RKO cells, all the other sequences have EC50 values<10 nM, and the effects of the sequences MD8-21-hm5# and MD8-21-h5# are relatively good. In the mouse-derived cell lines, EC50<10 nM only occurs for MD8-21-hm5# in mouse prostate cancer (RM-1) cells and colorectal cancer (MC38) cells as well as MD8-21-hm4# in colorectal cancer (MC38) cells. Therefore, based on the above data, the human-mouse homologous sequence MD8-21-hm5# is preferably used for pharmacodynamic evaluation of animal models in vivo.

### Example 4

In Vitro Screening of siRNA Sequences Targeting TGF-β1 Gene (Cellular Level)

For the target gene TGF-β1, due to its wide expression, only a small number of tumor cell lines were selected for the in vitro screening (cellular level), including bile duct cancer, breast cancer, lung cancer, pancreatic cancer, colorectal cancer and glioma cells. The experiment was performed using the same method as used for MyD88. The TGF-β1 siRNA sequences shown in Table 1 are the candidate sequences obtained after screening.

### Example 5

Evaluation of Knockdown Effects of Candidate siRNA Sequences on Target Gene Protein Levels in In-Vitro Cell Lines

The target gene knockdown effects of siRNA sequences on the targets TGF-β1 and MyD88 at the cellular level were evaluated by Western Blot. The procedures were as follows: on The day before the initiation of the experiment, the cells were inoculated in a 6-well plate at 1×10⁶ cells per well, and cultured overnight. On the second day, siMyD88 and siTF1 were transfected into the cells at 100 nM and 10 nM respectively. After transfection for 48 h, the cells were collected and transferred to centrifuge tubes. RIPA lysate was added to each tube to extract total cell protein, and then the total cell protein concentration was quantified by a BCA protein quantification method. Western Blot analysis was performed on all protein samples using the following antibodies: MyD88 primary antibody (ab219413, 1:1000), TGF-β1 primary antibody (ab215715, 1:1000), Goat Anti-Rabbit IgG H&L (HRP) (ab205718, 1:10000). After the experiment, the ChemiDoc™ MP Imaging System (BIO-RAD) was used to capture the results.

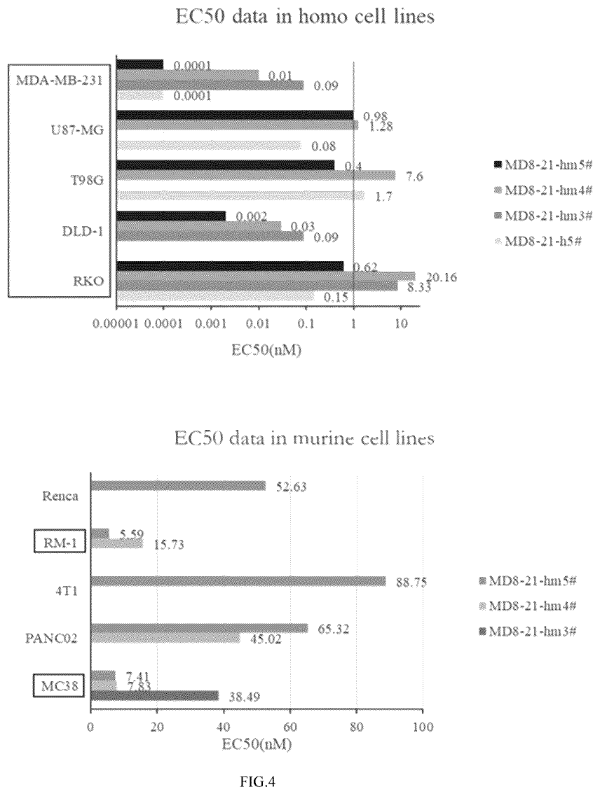

Western Blot analysis of protein expression in candidate MyD88 siRNA (MD8-21-hm5#) plus TGF-β1 siRNA (TF1-21-h1#) in three cell lines in in-vitro human breast cancer cell line (MDA-MB-231) (FIG. 5A), human colon cancer cell line (RKO) (FIG. 5B) and mouse prostate cancer (RM-1) cells (FIG. 5C). FIG. 5 shows that candidate MyD88 siRNA (MD8-21-hm5#) has obvious knockdown effect in all the three cell lines; TGF-β1 siRNA (TF1-21-h1#) has obvious knockdown effect in the RKO cell line; and candidate MyD88 siRNA (MD8-21-hm5#) plus TGF-β1 siRNA (TF1-21-h1#) has significant knockdown effect on the corresponding target protein in all the three cell lines.

### Example 6

In Vitro Evaluation of Effects of Candidate siRNA on Cell Migration in Human Colorectal Cancer Epithelial Cell Line DLD-1

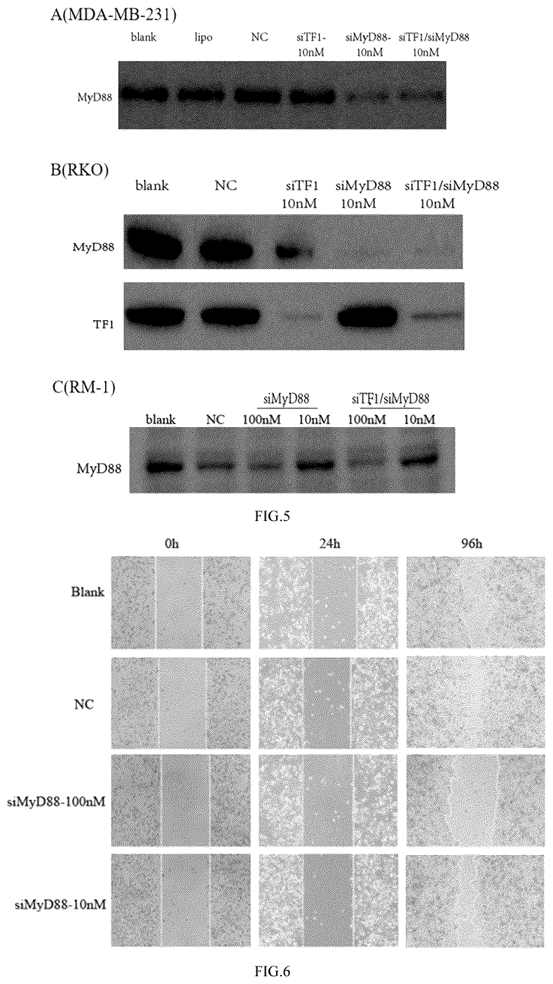

On the day before the initiation of the experiment, a 12-well plate was prepared, a 2-well culture-insert for wound healing (ibidi, 80209) was placed in the center of the culture dish, and cells were inoculated into the 2-well culture-insert at 1×10⁴ cells per well and cultured overnight. On the second day, siMyD88 was transfected into the cells at 100 nm and 10 nM, and the transfection experiment was completed. After transfection for 24 h, the 2-well culture-insert for wound healing was gently removed with tweezers (to avoid cell shedding), and the 12-well plate was added with a medium containing 1% serum to continue cell culture. At the same time, a photograph was taken under the microscope as the 0 h cell migration data. The culture was continued for 24 h and 96 h, and the cell migration was recorded under the microscope. The cell migration was determined by comparing the wound distance at the initial scratch (0 h) versus at the later observation points (24 h and 96 h). The results show (FIG. 6) that the candidate MyD88 siRNA monotherapy (MD8-21-hm5#) has a significant inhibitory effect on the in vitro migration ability of the DLD-1 cells at 96 h, and has a dose-dependent effect.

### Example 7

In Vitro Evaluation of Effects of Candidate siRNA on IL-6 Cytokine Secretion by RM-1 Cells of Mouse Prostate Cancer Cell Line

On The day before the initiation of the experiment, the cells were inoculated into a 24-well plate at 1×10⁵ cells per well and cultured overnight. On the second day, siMyD88 and siTF1 were transfected into the cells at 100 nM and 10 nM, respectively. After transfection for 48 h, the supernatant of cell culture was taken for Elisa detection. The cell culture supernatant was centrifuged at 300×g for 10 min to remove pellets (usually no pellets), immediately detected or subpackaged, and stored at −20° C. For the assay procedure, refer to Mouse IL-6 ELISA Kit (MULTISCIENCES, EK206/3-96). The procedure was run with 2 replicate wells for the standard and 3 replicate wells for the experimental group. At the end of the experiment, ELISA Calc software was used to draw a standard curve, and the concentration of the experimental group was calculated. The results show (FIG. 7) that IL-6 in the RM-1 cells is significantly decreased in both the high-concentration single-drug group and the combined double-drug group after transfection for 48 h. The results show that both siMyD88 and siTF1 could inhibit the expression of downstream IL-6, and have a dose-dependent effect.

### Example 8

### Preparation and Identification of Nano Drug Formulations

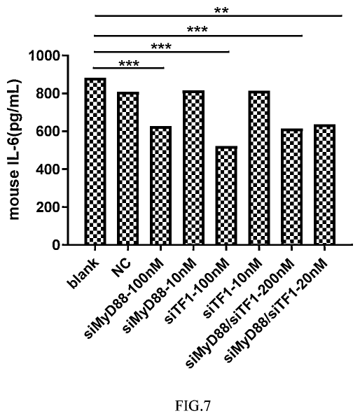

Through in vitro screening, the screening of siRNA sequences for the targets TGF-β1 and MyD88 at the cellular level was completed. Then, the candidate siRNA sequences of the two targets were used to prepare nano drug formulations. In this example, MD8-21-hm5# and TF1-25-hm5# were selected and mixed at a mass ratio of 1:1. The mixture together with the polypeptide carrier (HKP or HKP(+H)) formed a stable nanoparticle formulation (refer to patent CN 112703196 A). The prepared nanoparticle formulation was labeled STP500 for in vivo pharmacodynamic validation. In this example, reference could be made to Table 2 and Table 3 for the information about excipients and the parameters of the formulations. The finished products are shown in FIG. 8. The upper pictures are lyophilized formulations (MyD88 siRNA formulation on the left, MyD88 siRNA+TGF-β1 siRNA formulation on the right), and the lower pictures are the redissolved formulations (MyD88 siRNA formulation on the left, MyD88 siRNA+TGF-β1 siRNA formulation on the right).

TABLE 2
Preparation of candidate MyD88 siRNA and MyD88 siRNA plus TGF-β1
siRNA formulation (STP500) in related experiments of the present
disclosure.

| Drug | Excipient information | | siRNA source information | |
|---|---|---|---|---|
| demand | Name | Lot number | Name | N/P |
| MD8 | HKP(+H) | 19082001 | MD8-21-hm5# | 2.5/1 |
| MD8/TF1 | HKP(+H) | 19082001 | MD8-21-hm5# + TF1-25-hm5# | 2.5/1 |

N/P in the table refers to the N/P of polypeptide carrier to siRNA source.

TABLE 3
Parameters of candidate MyD88 siRNA and MyD88 siRNA plus TGF-β1 siRNA
formulation in related experiments of the present disclosure.

| | Lyophilized sample | | | | Redissolved sample | | | |
|---|---|---|---|---|---|---|---|---|
| | Size | | Zeta | UV | Size | | Zeta | UV |
| Sample | (nm) | PDI | (mv) | (ng/μl) | (nm) | PDI | (mv) | (ng/μl) |
| MD8 | 100.2 | 0.067 | 21.0 | 554.65 | 109.8 | 0.122 | 23.6 | 526.81 |
| MD8/TF1 | 82.65 | 0.104 | 22.8 | 558.07 | 108.3 | 0.197 | 22.3 | 546.56 |

### Example 9

### In Vivo Pharmacodynamic Validation (Mouse Subcutaneous Tumor Graft Model of Colorectal Cancer MC38 Cells)

Based on the data from previous in-vitro experiments, two siRNAs (MD8-21-hm5# and TF1-25-hm5# in this example) were mixed at a mass ratio of 1/1, and encapsulated the mixture into the HKP(+H) (histidine-lysine polymer) carrier through patented PNP technology to prepare the nanoparticle formulation (the nano drug formulation STP500 prepared in Example 8). The tumor growth inhibitory activity of the candidate siRNA sequence was validated by a mouse tumor xenograft model (colorectal cancer cells MC38).

C57BL/6 mice were each inoculated subcutaneously with 1×10⁶ MC38 cells per mouse, and randomized groups when the mean tumor volume reached about 100 mm³. The dosing regimen was as follows: for STP500, 2 mg/kg, intravenously administered (i.v.) via the tail vein, every 2 days (Q2D); for Small nucleic acid monotherapy MD8 (MyD88 siRNA, also known as siMyD88), 1 mg/kg, intravenously administered (i.v.) via the tail vein, every 2 days (Q2D); and for the positive control group (Sorafenib), 30 mg/kg, intragastrically administered p.o., every day (QD). After the first dose, the weight and tumor volume of the mice were recorded every 2 days, and the tumor volume was calculated by V=0.5×(Dmax×Dmin²), where Dmax refers to the longest diameter of the tumor, and Dmin refers to the shortest diameter of the tumor. A tumor volume curve was drawn. At the end of the experiment, the mice were euthanized, and tumors were removed and weighed. TGItw (percentage change in tumor weight) was calculated by:

$$TGItw = ( 1 - \frac{mean {TW}_{treat}}{mean {TW}_{vehicle}} ) \times 100 \%$$

Mean TWₜᵣₑₐₜ: the mean tumor weight of the mice in the treatment group at the end of treatment;
Mean TW_{vehicle}: the mean tumor weight of mice in the vehicle group at the end of treatment.

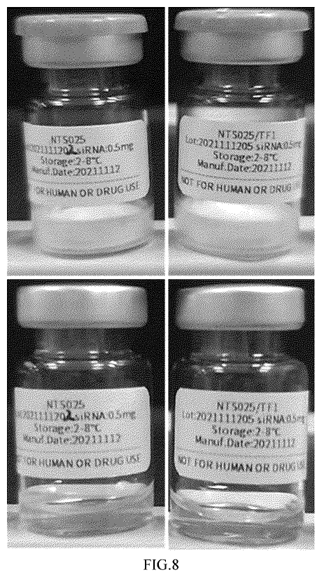

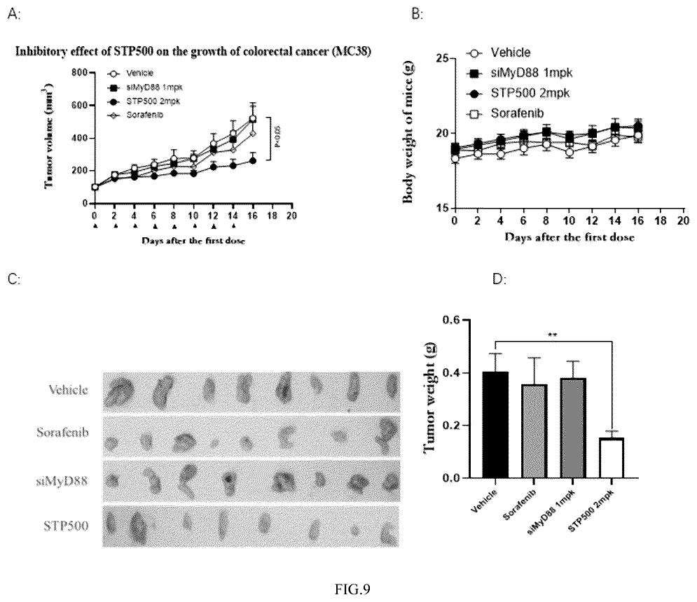

The results are shown in FIG. 9. The results show that compared with the control group, MyD88 and TGF-β1 combined treatment group (MD8+TF1) nanoparticle drug can significantly inhibit the growth of mouse colorectal cancer tumor graft (MC38 cell line) after 8 doses, and its effect is better than other groups (MyD88 siRNA monotherapy group (MD8)) and the positive control group (Sorafenib). In addition, compared with the model group (Vehicle), the tumor volume in the combined treatment group was reduced with a statistical difference (*P<0.05) (FIG. 9A). The tumor weight of the combined treatment group was reduced with a statistically significant difference (**P<0.01) (FIG. 9D). The tumor growth inhibition of the candidate combined treatment group (STP500) was 62% compared with the control group (Vehicle) (FIG. 9C, FIG. 9D, Table 4), and the difference in tumor weight was statistically significant (*P<0.05).

TABLE 4
Tumor growth inhibition of siMD8 monotherapy and STP500
in colorectal cancer (MC38) tumor graft model
Tumor weight (g)

| | Mean | SEM | TGItw |
|---|---|---|---|
| STP500 2 mpk | 0.152857 | 0.024041 | 62.46% |
| siMD8 1 mpk | 0.381429 | 0.059035 | 6.32% |
| Sorafenib | 0.357143 | 0.094046 | 12.28% |
| Vehicle | 0.407143 | 0.062106 | |

### Example 10

### In Vivo Pharmacodynamic Validation (Mouse Subcutaneous Tumor Xenograft Model of Gastric Cancer MKN45 Cells)

Next, another tumor graft model was established to further validate the pharmacodynamic evaluation of candidate drug on the growth of mouse tumor xenograft of human gastric cancer MKN45 cells. The nano drug formulation was prepared by the method described above. MKN45 subcutaneous tumor-bearing mice were modeled as follows: BALB/c-Nude mice were each inoculated subcutaneously with 5×10⁶ MKN45 cells. Randomization was performed when the mean tumor volume reached about 100-120 mm³. The dosing regimen was as follows: for STP500, 3 mg/kg, intravenously administered (i.v.) via the tail vein, every 2 days (Q2D); for the Positive control group (Oxaliplatin), 7.5 mg/kg, intraperitoneally administered (i.p.) every day (QD). After the first dose, the weight and tumor volume of the mice were recorded every 2 days, and the tumor volume was calculated by V=0.5×(Dmax×Dmin²), where Dmax refers to the longest diameter of the tumor, and Dmin refers to the shortest diameter of the tumor. A tumor volume curve was drawn at the end of the experiment. The mice were euthanized, and tumors were removed and weighed. TGItw (percentage change in tumor weight) was calculated by:

$$TGItw = ( 1 - \frac{mean {TW}_{treat}}{mean {TW}_{vehicle}} ) \times 100 \%$$

Mean TWₜᵣₑₐₜ: the mean tumor weight of the mice in the treatment group at the end of treatment;
Mean TW_{vehicle}: the Mean tumor weight of mice in the Vehicle group at the end of treatment.

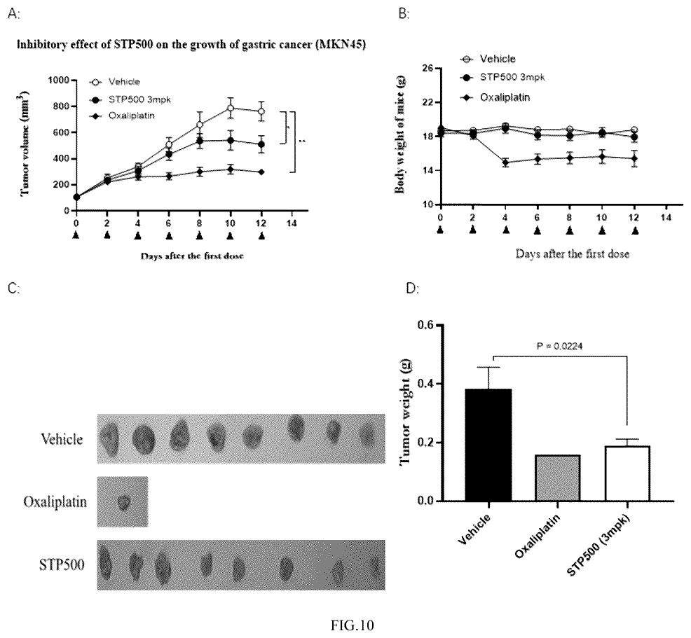

The results are shown in FIG. 10. The results show that compared with the control group, MyD88 and TGF-β1 combined treatment group (STP500) nanoparticle drug can significantly inhibit the growth of human gastric cancer tumor graft (MKN45 cell line) after 7 doses (FIG. 10A). At this dosage, siRNA nanoparticle drug had no significant effect on the weight of mice. In contrast, although the positive drug Oxaliplatin had better tumor inhibition effect, only one tumor sample was taken at the end of the experiment. In addition, it also showed certain toxicity, and the weight of mice in the group decreased (FIG. 10B). Compared with the control group (Vehicle), the tumor growth inhibition of the candidate combined treatment group (STP500) was 50% (FIG. 10C, FIG. 10D, Table 5), and the difference in tumor weight was statistically significant (*P<0.05).

TABLE 5
Tumor growth inhibition of STP500 in gastric
cancer (MKN45) tumor xenograft model.
Tumor weight (g)

| | Mean | SEM | TGItw |
|---|---|---|---|
| STP500 3 mpk | 0.19 | 0.06 | 50% |
| Oxaliplatin | 0.16 | NA | 58% |
| Vehicle | 0.39 | 0.20 | |

### Example 11

### In Vivo Pharmacodynamic Validation (Mouse Subcutaneous Tumor Graft Model of Prostate Cancer RM-1 Cells)

Next, another tumor graft model was established to further validate the pharmacodynamic evaluation of candidate drug on the growth of mouse tumor graft of mouse prostate cancer RM-1 cells. The nano drug formulation was prepared by the method described above. RM-1 subcutaneous tumor-bearing mice were modeled as follows: C57BL/6J mice were each inoculated subcutaneously with 5×10⁶ cells. The mice were grouped on the basis of random numbers when the mean tumor volume reached about 100-120 mm³. The dosing regimen was as follows: for STP500, 3 mg/kg, intravenously administered (i.v.) via the tail vein, every 2 days (Q2D); for The positive control (docetaxel), 15 mg/kg, intraperitoneally administered (i.p.), every week (QW). After the first dose, the weight and tumor volume of the mice were recorded every 2 days, and the tumor volume was calculated by V=0.5×(Dmax×Dmin²), where Dmax refers to the longest diameter of the tumor, and Dmin refers to the shortest diameter of the tumor. A tumor volume curve was drawn at the end of the experiment. The mice were euthanized, and tumors were removed and weighed. TGItw (percentage change in Tumor weight) was calculated by:

$$TGItw = ( 1 - \frac{mean {TW}_{treat}}{mean {TW}_{vehicle}} ) \times 100 \%$$

Mean TWₜᵣₑₐₜ: the mean tumor weight of the mice in the treatment group at the end of treatment;
Mean TW_{vehicle}: the Mean tumor weight of mice in the Vehicle group at the end of treatment.

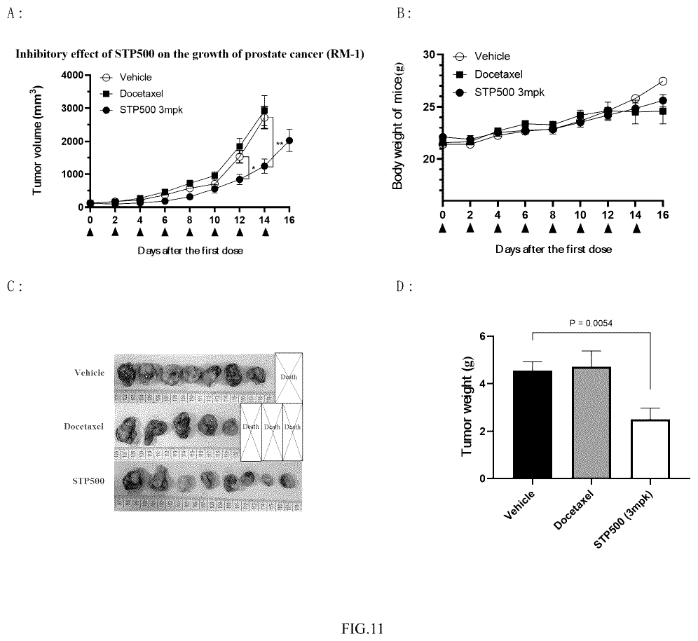

The results are shown in FIG. 11. The results show that compared with the control group, MyD88 and TGF-β1 combined treatment group (STP500) nanoparticle drug can significantly inhibit the growth of mouse-derived prostate cancer (RM-1 cell line) after 8 doses (FIG. 11A). On the 12^{th} day after the first dose, the tumor volume in the STP500 treatment group was significantly reduced compared with that in the vehicle group (*P<0.05). On the 14th day after the first dose, the tumor volume in the STP500 treatment group was significantly reduced compared with that in the Vehicle group (*P<0.01). At this dosage (3 mg/kg, Q2D), siRNA nanoparticle drug had no significant effect on the weight of mice. In contrast, Docetaxel, a commonly used chemotherapy drug, did not show a tumor-killing effect in this experiment, indicating certain clinical limitations. There was no significant difference in weight change of mice in the group (FIG. 11B). Compared with the control group (vehicle), The tumor growth inhibition of the candidate combined treatment group (STP500) was 45% (FIG. 11C, FIG. 11D, Table 6), and the difference in tumor weight was statistically significant (**P<0.01).

TABLE 6
Tumor growth inhibition of STP500 in prostate
cancer (RM-1) tumor graft model.
Tumor weight (g)

| | Mean | SEM | TGItw |
|---|---|---|---|
| STP500 | 2.51 | 0.47 | 45% |
| Docetaxel | 4.72 | 0.66 | −4% |
| Vehicle | 4.55 | 0.37 | |

### Example 12

### In Vivo Pharmacodynamic Validation (Mouse Subcutaneous Tumor Graft Model of Colorectal Cancer MC38 Cell Lines—Independent Parallel Trials)

In another set of independent parallel trials, the pharmacodynamic evaluation of candidate drug STP500 was repeatedly tested in the subcutaneous tumor graft of colorectal cancer MC38 cell line, and compared the transcriptome sequencing data of the treatment group (STP500) and the siRNA drug negative control group (NC). The nano drug formulation was prepared by the method described above. MC38 subcutaneous tumor-bearing mice were modeled as follows: C57BL/6 mice were each inoculated subcutaneously with 1×10⁶ MC38 cells, and randomized when the mean tumor volume reached about 100 mm³. The dosing regimen was as follows: for STP500, 2 mg/kg, intravenously administered (i.v.) via the tail vein, every 3 days (Q3D); for the Positive control group (mPD-L1, PD-L1 antibody), 5 mg/kg, intraperitoneally administered (i.p.), every 3 days (Q3D). After the first dose, the survival and tumor volume of the mice were recorded 3 times a week, and the tumor volume was calculated by V=0.5×(Dmax×Dmin²), where Dmax refers to the longest diameter of the tumor, and Dmin refers to the shortest diameter of the tumor. A tumor volume curve was drawn at the end of the experiment. The mice were euthanized, and tumors were removed and weighed.

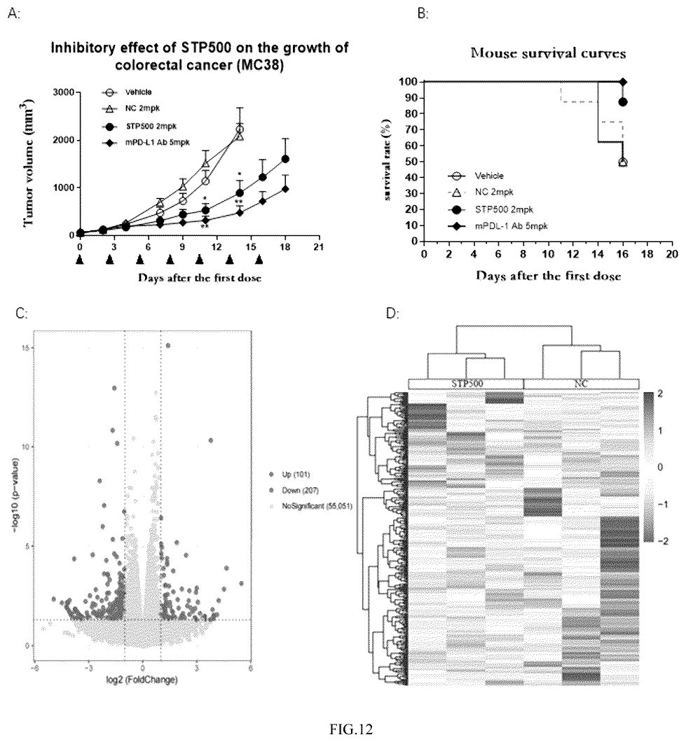

The results are shown in FIG. 12. The results show that compared with the control group, MyD88 and TGF-β1 combined treatment group (STP500) nanoparticle drug can significantly inhibit the growth of mouse-derived colorectal cancer (MC38 cell line) after 7 doses (FIG. 12a), which was consistent with our previous conclusions. On the 14th day after the first dose, the tumor volume in the STP500 treatment group was significantly reduced compared with that in the Vehicle group (*P<0.05). A survival curve of mice in the group also showed the corresponding conclusions (FIG. 12B).

At the end of the experiment (on the 18^{th} day after the first dose), 3 mice were randomly selected from the STP500 treatment group and the small nucleic acid drug negative control group (NC) to conduct transcriptome sequencing of tumor samples. The sequencing work was completed by Suzhou GENEWIZ Biotechnology Co., LTD. Then, we processed and analyzed the sequencing data, and used Hisat2 (v2.0.1) to index the genome sequence. Finally, the clean data was compared with the reference genome using the software Hisat2 (v2.0.1). A text in fasta format was converted from a known gff annotation file and properly indexed. HTSeq (v0.6.1) then used this file as a reference gene file to estimate gene and subtype expression levels from the paired-end clean data. Differential expression analysis was performed by using DESeq2 Bioconductor package, a model based on a negative binomial distribution. Data-driven prior distributions were used to estimate the variance and log fold change, where |log 2(FoldChange)|>1 and p-value<0.05 were considered differentially expressed genes. A STRING database (https://string-db.org/) was used to construct a protein Interaction network of differentially expressed genes, with Interaction Score>0.4 as the threshold. The results were imported into Cytoscape software (v3.9.1) for analysis and mapping of betweenness centrality using Cytoscape's plugin CytoNCA. The R package clusterProfiler(v4.4.4) was used for GO (Gene Ontology) functional enrichment analysis and KEGG (Kyoto Encyclopedia of Genes and Genomes) analysis of differentially expressed genes, and the R package ggplot² (v3.3.6) was used to plot the results.

The statistical results are shown in FIGS. 12 (C & D). Compared with the expression of genes in the NC group, we detected 101 up-regulated genes and 207 down-regulated genes in samples from the STP500 treatment group (FIG. 12C). For example, the expression of some tumor-related genes (Claudin-5, Vtn, Ambp, Col1a1, etc.) was down-regulated, while the expression of immune-activation-related genes (CD28, etc.) was increased. The expressions of the up-regulated and down-regulated gene in the samples from the two groups are shown in the heat map (FIG. 12D). Table 7 lists part of the candidate genes and their related biological functions. The transcriptome data provide certain support for subsequent studies on target gene related pathways and the interaction between this target and other targets, and provide data support for the future development of other related small nucleic acid drugs or combination treatment strategies.

TABLE 7
Transcriptome analysis data—Representative candidate target-related
genes.

| | | | Up- |
|---|---|---|---|
| | | | regulated/ |
| | | | down- |
| Gene | name | Biological functions | regulated |
| Cldn5 | Claudin 5 | It is associated with lymph node | Down- |
| | | metastasis in colorectal cancer. | regulated |
| Vtn | Vitronectin | It is present in plasma and extracellular | Down- |
| | | matrix. Vitronectin binds to specific cell | regulated |
| | | surface receptors such as integrin αVβ3 | |
| | | and αVβ5 through the mediation by the | |
| | | Arg-Gly-Asp (RGD) sequence. | |
| | | Vitronectin can promote the attachment, | |
| | | extension and proliferation of | |
| | | endothelial cells, and promote the | |
| | | differentiation of a variety of normal | |
| | | cells and cancer cells. Vitronectin can be | |
| | | used for cell migration study. | |
| Col 1a1 | Type I | Type I collagen, the most abundant | Down- |
| | collagen | protein in the body, is produced by | regulated |
| | | fibroblasts and is found primarily in | |
| | | bone, tendons, and skin. In its normal | |
| | | form, collagen is a heterotrimer | |
| | | composed of two α1 chains (Col1a1) | |
| | | and one α2 chain (Col1a2) that assemble | |
| | | to form a triple helix as part of the | |
| | | extracellular matrix. However, during | |
| | | the study on human pancreatic cancer | |
| | | cell lines, researchers have found that | |
| | | these cells could produce atypical forms | |
| | | of collagen. | |
| CD28 | T-cell- | CD28 is a costimulatory molecule | Up- |
| | specific | expressed on the surface of T | regulated |
| | surface | lymphocytes, which plays an important | |
| | glycoprotein | role in the activation of T cells. It binds | |
| | CD28 | to B7 molecules on APC (antigen- | |
| | | presenting cells), mediates costimulation | |
| | | of T cells and promotes their survival, | |
| | | proliferation, and cytokine production. | |
| Nos3 | Endothelial | Endothelial nitric oxide synthase is | Down- |
| | nitric oxide | mainly distributed in the endocardium of | regulated |
| | synthase | coronary vessels and the cardiac cavity | |
| | | surfaces. Its main function is to | |
| | | participate in the metabolism of arginine | |
| | | and proline and catalyze the production | |
| | | of nitric oxide (NO). It has been | |
| | | reported that the increased activity of | |
| | | NOS is associated with tumor | |
| | | angiogenesis. | |
| Epcam | Epithelial | Epcam, also known as CD326, vascular | Down- |
| | cell | endothelium-associated molecule, is a | regulated |
| | adhesion | 40 kDa transmembrane glycoprotein | |
| | molecule | encoded by GA-732-2 gene. As an | |
| | | homophilic calcium-independent | |
| | | adhesion molecule between endothelial | |
| | | cells, it plays a role in epithelial | |
| | | carcinogenesis. | |

CytoNCA: a cellular landscape plug-in for centrality analysis and evaluation of protein interaction networks;
clusterProfiler: R package for comparing biological topics between gene clusters.

Based on the data from the above examples, the combination drug (STP500), which is encapsulated by the PNP technology and has siRNA co-targeting MyD88 (MD8) and TGF-β1 (TF1) introduced therein, has potential application prospects in the treatment of gastrointestinal cancers (such as colorectal cancer, gastric cancer) and prostate cancer.

The above examples are only used to illustrate the technical idea and characteristics of the present disclosure, and are intended to enable persons familiar with the technology to understand the content of the present disclosure and implement it accordingly. The scope of protection of the present disclosure shall not be limited thereto. Any equivalent variation or modification made in accordance with the spirit of the present disclosure shall be covered by the scope of protection of the present disclosure.

1. A RNA interference-based pharmaceutical composition comprising an active substance and a pharmaceutically acceptable carrier, wherein the active substance comprises a first active substance capable of inhibiting and silencing MyD88 gene expression and a second active substance capable of inhibiting and silencing TGF-β1 gene expression.

2. The RNA interference-based pharmaceutical composition according to claim 1, wherein the first active substance is one or more of siRNA molecules, miRNA molecules, or antisense oligonucleotide molecules capable of binding to mRNA encoding MyD88 protein and inhibiting its expression; and/or the second active substance is one or more of siRNA molecules, miRNA molecules, or antisense oligonucleotide molecules capable of binding to mRNA encoding TGF-β1 protein and inhibiting its expression.

3. The RNA interference-based pharmaceutical composition according to claim 2, wherein the first active substance is a siRNA molecule targeting the MyD88 gene, comprising one or more sets of the following oligonucleotides:
sense strand: CGUUGUAGGAGGAAUCUGUdTdT (SEQ ID NO: 1), antisense strand: ACAGAUUCCUCCUACAACGdTdT (SEQ ID NO: 2);
sense strand: GAGGAAUCUGUGCUCUACUdTdT (SEQ ID NO: 3), antisense strand: AGUAGAGCACAGAUUCCUCdTdT (SEQ ID NO: 4);
sense strand: GGAAUCUGUGCUCUACUUAdTdT (SEQ ID NO: 5), antisense strand: UAAGUAGAGCACAGAUUCCdTdT (SEQ ID NO: 6);
sense strand: GCUCUACUUACCUCUCAAUdTdT (SEQ ID NO: 7), antisense strand: AUUGAGAGGUAAGUAGAGCdTdT (SEQ ID NO: 8);
sense strand: GCAUACACACGUUUUUCUAdTdT (SEQ ID NO: 9), antisense strand: UAGAAAAACGUGUGUAUGCdTdT (SEQ ID NO: 10);
sense strand: CCCAAUGUACCAGUAUUUAdTdT (SEQ ID NO: 11), antisense strand: UAAAUACUGGUACAUUGGGdTdT (SEQ ID NO: 12);
sense strand: GCUUAAACUCACACAACAAdTdT (SEO ID NO: 13), antisense strand: UUGUUGUGUGAGUUUAAGCdTdT (SEQ ID NO: 14);
sense strand: GACCCUAAAUCCAAUAGAAdTdT (SEQ ID NO: 15), antisense strand: UUCUAUUGGAUUUAGGGUCdTdT (SEQ ID NO: 16);
sense strand: CUUGUUGAGGCAUUUAGCUdTdT (SEQ ID NO: 17), antisense strand: AGCUAAAUGCCUCAACAAGdTdT (SEQ ID NO: 18);
sense strand: GGCAUCUUCUACAUGUUUUdTdT (SEQ ID NO: 19), antisense strand: AAAACAUGUAGAAGAUGCCdTdT (SEQ ID NO: 20);
sense strand: CUGAGAAAAGCCGAUAUUUdTdT (SEQ ID NO: 21), antisense strand: AAAUAUCGGCUUUUCUCAGdTdT (SEQ ID NO: 22);
sense strand: GAGAAGCCUUUACAGGUGGdTdT (SEQ ID NO: 23), antisense strand: CCACCUGUAAAGGCUUCUCdTdT (SEQ ID NO: 24);
sense strand: AGGAGAUGAUCCGGCAACUdTdT (SEQ ID NO: 25), antisense strand: AGUUGCCGGAUCAUCUCCUdTdT (SEQ ID NO: 26);
sense strand: CAGAGCAAGGAAUGUGACUdTdT (SEQ ID NO: 27), antisense strand: AGUCACAUUCCUUGCUCUGdTdT (SEQ ID NO: 28);
sense strand: GCAAGGAAUGUGACUUCCAdTdT (SEQ ID NO: 29), antisense strand: UGGAAGUCACAUUCCUUGCdTdT (SEQ ID NO: 30);
sense strand: GAAUGUGACUUCCAGACCAdTdT (SEQ ID NO: 31), antisense strand: UGGUCUGGAAGUCACAUUCdTdT (SEQ ID NO: 32);
and/or,
the second active substance is a siRNA molecule targeting the TGF-β1 gene, comprising one or more sets of the following oligonucleotides:
sense strand: CCCAAGGGCUACCAUGCCAACUUCU (SEQ ID NO: 33), antisense strand: AGAAGUUGGCAUGGUAGCCCUUGGG (SEQ ID NO: 34);
sense strand: AACUAUUGCUUCAGCUCCAdTdT (SEQ ID NO: 35), antisense strand: UGGAGCUGAAGCAAUAGUUdTdT (SEQ ID NO: 36);
sense strand: GCAGAGUACACACAGCAUAdTdT (SEQ ID NO: 37), antisense strand: UAUGCUGUGUGUACUCUGCdTdT (SEQ ID NO: 38).

4. The RNA interference-based pharmaceutical composition according to claim 1, wherein the carrier is one or more of a polycation binders, a cationic liposome, a cationic micelle, a cationic polypeptide, a cationic polyacetal, a grafted hydrophilic polymer, a polysaccharide molecule, a polyvesicle, an antibody, a polypeptide molecule, or an aptamer.

5. The RNA interference-based pharmaceutical composition according to claim 42, wherein the carrier is a histidine-lysine polymer.

6. The RNA interference-based pharmaceutical composition according to claim 5, wherein the carrier is an H3K4b type histidine-lysine polymer.

7. The RNA interference-based pharmaceutical composition according to claim 5, wherein the carrier is HKP or HKP(+H).

8. A drug for treating one or more of colorectal cancer, gastric cancer, and prostate cancer, comprising a RNA interference-based pharmaceutical composition as described in claim 1.

9. The drug according to claim 8, wherein the feed mass ratio of the first active substance to the second active substance is 1:0.8 to 1.2.

10. The drug according to claim 8, wherein the N/P ratio of the carrier to the active substance is 2/1-6/1.

11. The drug according to claim 8, wherein the drug is in the form of nanoparticles.

12. The drug according to claim 8, wherein the drug is administered by subcutaneous injection and/or intravenous injection.

13. The drug according to claim 8, is-characterized in that wherein the drug is administered to mammals.

14. The drug according to claim 8, wherein the first active substance is a siRNA molecule targeting the MyD88 gene, comprising the following sequences: sense strand: GAAUGUGACUUCCAGACCAdTdT (SEQ ID NO: 31), antisense strand: UGGUCUGGAAGUCACAUUCdTdT (SEQ ID NO: 32);
the second active substance is a siRNA molecule targeting the TGF-β1 gene, comprising the following sequences: sense strand: CCCAAGGGCUACCAUGCCAACUUCU (SEQ ID NO: 33), antisense strand: AGAAGUUGGCAUGGUAGCCCUUGGG (SEQ ID NO: 34).

15. A method for treating colorectal cancer, gastric cancer, prostate cancer, comprising administering an RNA interference-based pharmaceutical composition to a subject, wherein the RNA interference-based pharmaceutical composition is as claimed in claim 1.

16. The RNA interference-based pharmaceutical composition according to claim 1, wherein the first active substance is a siRNA molecule targeting the MyD88 gene, comprising the following sequences: sense strand: GAAUGUGACUUCCAGACCAdTdT (SEQ ID NO: 31), antisense strand: UGGUCUGGAAGUCACAUUCdTdT (SEQ ID NO: 32);
the second active substance is a siRNA molecule targeting the TGF-β1 gene, comprising the following sequences: sense strand: CCCAAGGGCUACCAUGCCAACUUCU (SEQ ID NO: 33), antisense strand: AGAAGUUGGCAUGGUAGCCCUUGGG (SEQ ID NO: 34).

17. The method for treating colorectal cancer, gastric cancer, prostate cancer according to claim 15, wherein the first active substance is a siRNA molecule targeting the MyD88 gene, comprising the following sequences: sense strand: GAAUGUGACUUCCAGACCAdTdT (SEQ ID NO: 31), antisense strand: UGGUCUGGAAGUCACAUUCdTdT (SEQ ID NO: 32);
the second active substance is a siRNA molecule targeting the TGF-β1 gene, comprising the following sequences: sense strand: CCCAAGGGCUACCAUGCCAACUUCU (SEQ ID NO: 33), antisense strand: AGAAGUUGGCAUGGUAGCCCUUGGG (SEQ ID NO: 34).
